(19) United States
(12) Patent Application Publication
(10) Pub. No.: US 2026/0232570 A1
(43) Pub. Date: Aug. 13, 2026

(54) COSMETIC COMPOSITION AND METHOD FOR MANUFACTURING THE SAME

(71) Applicant: INDUSTRIAL TECHNOLOGY RESEARCH INSTITUTE, Hsinchu (TW)

(72) Inventors: Ting-Yu SHIH, Taipei City (TW); Lu-Chih WANG, Hsinchu City (TW); Yu-Chun LIU, Taichung City (TW); Yuan-Kun YU, Yilan City (TW); Yi-Ting HSIEH, Zhudong Township (TW)

(73) Assignee: INDUSTRIAL TECHNOLOGY RESEARCH INSTITUTE, Hsinchu (TW)

(21) Appl. No.: 19/432,592

(22) Filed: Dec. 24, 2025

(30) Foreign Application Priority Data
Dec. 31, 2024 (TW) 113151706

(51) Int. Cl. A61K 8/9794; A61Q 19/08

(52) U.S. Cl. CPC A61K 8/9794; A61Q 19/08; A61K 2800/805

(57) ABSTRACT

A method for manufacturing a cosmetic composition is provided. The method includes preparing a pineapple leaf extract. Steps for preparing the pineapple leaf extract include: providing pineapple leaves; performing a size reduction step on the pineapple leaves to obtain a pineapple leaf powder; performing an extraction step on the pineapple leaf powder using an aqueous alcohol solution, and collecting an extract resulting therefrom; and performing a sterilization step on the extract to obtain the pineapple leaf extract.

This application claims the benefit of Taiwan Patent application Serial No. 113151706, filed on Dec. 31, 2024, the disclosure of which is incorporated by reference herein in its entirety.

### TECHNICAL FIELD

The disclosure relates to a cosmetic composition and a method for manufacturing the same.

### BACKGROUND

Pineapple is one of the widely popular fruits; however, the large-scale cultivation of pineapple has resulted in issues regarding the disposal of pineapple leaf waste. Pineapple leaves are not easily processed by composting, which in turn leads to the occupation of farmland resources. If pineapple leaves are disposed of by incineration, problems of air pollution and carbon emissions may arise.

Therefore, there is still an urgent need to find an environmentally friendly solution for processing pineapple leaf waste.

### SUMMARY

According to one embodiment, a method for manufacturing a cosmetic composition is provided. The method comprises preparing a pineapple leaf extract. Steps for preparing the pineapple leaf extract include: providing pineapple leaves; performing a size reduction step on the pineapple leaves to obtain a pineapple leaf powder; performing an extraction step on the pineapple leaf powder using an aqueous alcohol solution, and collecting an extract resulting therefrom; and performing a sterilization step on the extract to obtain the pineapple leaf extract.

According to another embodiment, a cosmetic composition is provided. The cosmetic composition is manufactured by the method described above, wherein the cosmetic composition comprises 1 wt % to 5 wt % of the pineapple leaf extract and 95 wt % to 99 wt % of water. The pineapple leaf extract has a total polyphenol content of 150 mg GAE/100 g pineapple leaves and 550 mg GAE/100 g pineapple leaves.

### BRIEF DESCRIPTION OF THE DRAWINGS

FIG. 1 is a flowchart illustrating a method for manufacturing a pineapple leaf extract according to an embodiment of the present disclosure.

FIG. 2 illustrates the antioxidant activity percentages of pineapple leaf extracts in Examples C1 to C3 according to an embodiment of the present disclosure.

FIG. 3 illustrates the results of collagen secretion for Comparative Example D and Examples D1 to D3.

FIG. 4 illustrates the results of collagen secretion for Comparative Examples E1 to E3 and Example E1.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

Various embodiments will be described in more detail below with reference to the accompanying drawings. The descriptions and drawings are provided for illustrative purposes only and are not intended to be limiting. For the sake of clarity, some elements and/or symbols may be omitted in certain drawings. In addition, elements in the drawings may not be drawn to scale. It is contemplated that elements and features from one embodiment can be advantageously incorporated into another embodiment without further recitation.

The present disclosure is directed to a cosmetic composition and a method for manufacturing the same. The cosmetic composition includes 1 wt % to 5 wt % of pineapple leaf extract and 95 wt % to 99 wt % of water, wherein a total polyphenol content of a pineapple leaf extract is between 150 mg GAE/100 g pineapple leaves and 550 mg GAE/100 g pineapple leaves. “GAE” represents gallic acid equivalent, which is measured based on the weight of 100 g of the pineapple leaves.

Since the pineapple leaf extract of the present disclosure has a high total polyphenol content, the cosmetic composition can have excellent antioxidant activity and achieve superior cosmetic effects, such as anti-aging, collagen production, and the like.

According to some embodiments, the pineapple leaf extract includes one or more active ingredients, and the one or more active ingredients comprise total polysaccharides, ferulic acid, chlorogenic acid (CGA), p-coumaric acid (P-CA), or any combination thereof. For example, based on a weight of 100 g of the pineapple leaves, a weight of the total polysaccharides ranges from 1 g to 5 g, a weight of the ferulic acid ranges from 0.1 mg to 30 mg, a weight of the p-coumaric acid ranges from 0.1 mg to 30 mg, and a weight of the chlorogenic acid ranges from 0.1 mg to 30 mg. It should be understood that the active ingredients of the pineapple leaf extract of the present disclosure are not limited thereto. The weight of the total polysaccharides is measured by the phenol-sulfuric acid (PSA) total polysaccharide quantification method. The weights of ferulic acid, chlorogenic acid, and p-coumaric acid are determined by HPLC.

According to some embodiments, the pineapple leaf extract provides a cosmetic composition having enhanced antioxidant activity and/or a cosmetic composition having a capability to promote collagen secretion.

According to some embodiments, the present disclosure further provides a method for manufacturing a cosmetic composition. The method includes preparing a pineapple leaf extract. The steps of manufacturing the pineapple leaf extract include: providing pineapple leaves; performing a size reduction step on the pineapple leaves to obtain pineapple leaf powder; performing an extraction step on the pineapple leaf powder using an aqueous alcohol solution and collecting an extract resulting therefrom; and performing a sterilization step on the extract to obtain the pineapple leaf extract.

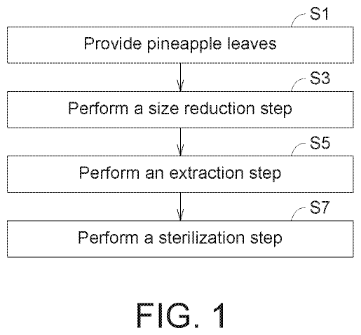

As shown in FIG. 1, a method for preparing a pineapple leaf extract according to an embodiment of the present disclosure includes the following sequential steps S1 to S7.

In step S1, pineapple leaves are provided as a raw material. The appearance, heavy metal residues, pesticide residues, or other inspection items of the pineapple leaves may be tested and should comply with relevant regulatory requirements.

In step S3, a size reduction step is performed, for example, using a high-speed centrifugal mill, to obtain an average particle size between 0.01 mm and 0.8 mm (e.g., 0.04 mm to 0.25 mm) under conditions of a rotation speed of 6000 rpm to 18000 rpm, a ring sieve size of 0.08 mm to 1 mm, and a temperature of 25±2° C., so as to form the pineapple leaf powder.

Optionally, before performing the size reduction step S3, a drying step (low-temperature drying process) may be performed on the pineapple leaves, for example, by using a cyclone dryer at 50° C. to 70° C. (e.g., 60° C.) with a feeding rate of 0.2 Kg s⁻¹. The pineapple leaves have a moisture content of not greater than 10% after the drying step (by weight percentage on a dry weight basis).

In step S5, an extraction step is performed. For example, the pineapple leaf powder may be extracted with an aqueous alcohol solution (e.g., 30% aqueous ethanol solution) for 1 hour, followed by a second extraction, and the resulting extract is collected. In the present embodiment, the aqueous alcohol solution consists of water and ethanol, wherein ethanol accounts for about 30 volume percentage (vol %) of the total volume of the aqueous alcohol solution, such as 25 vol % to 35 vol %. However, the types of alcohols and the ranges of volume percentages thereof in the present disclosure are not limited thereto. It should be understood that other suitable solvents may also be used for extraction.

In step S7, a sterilization step is performed on the extract. For example, a syringe filter with pore sizes of 0.45 μm and 0.22 μm may be used to perform two sterilization filtrations to obtain the pineapple leaf extract.

It should be understood that the method for preparing the pineapple leaf extract of the present disclosure may also include other steps, but is not limited thereto.

Compared with the comparative example that has not undergone the size reduction step, the pineapple leaf extract of an embodiment of the present disclosure can obtain pineapple leaf powder with a smaller particle size (e.g., between 0.1 mm and 0.5 mm) due to the size reduction treatment, thereby possessing a higher specific surface area and enhancing the extraction efficiency of organic solvents. Therefore, more active ingredients can be extracted during the subsequent extraction step, such as having higher contents of total polyphenols, chlorogenic acid, and p-coumaric acid, exhibiting better antioxidant activity and having a superior collagen secretion-promoting effect.

Compared with the comparative example using a conventional oven drying method for the drying step, the pineapple leaves of an embodiment of the present disclosure are formed using cyclone low-temperature drying, which can retain more active ingredients (such as p-coumaric acid and chlorogenic acid) to reduce the destruction of active ingredients by high temperatures. Therefore, the pineapple leaf extract prepared by the present disclosure possesses better cosmetic efficacy. For example, its total polyphenol content can be increased by about 50 times, and the antioxidant capacity can be further enhanced (increased by about 55% in the antioxidant test with ABTS reagent, and increased by about 20.15% in the antioxidant test with DPPH reagent; wherein the full English name of ABTS is 2,2′-azino-bis(3-ethylbenzothiazoline-6-sulfonic acid), and the full English name of DPPH is 2,2-diphenyl-1-picrylhydrazyl).

In order to make the above and other objects, features, and advantages of the present invention more comprehensible, several embodiments are listed below and described in detail as follows:

### Antioxidant Test

According to some embodiments, an ABTS radical scavenging assay may be performed on the pineapple leaf extract. Specifically, the ABTS radical scavenging assay can be used to evaluate the antioxidant capacity of a sample to be tested. ABTS undergoes an oxidation reaction after reacting with potassium persulfate (K₂S₂O₈) to form stable blue-green water-soluble ABTS·+radical ions, which have a maximum absorption peak at 734 nm. Therefore, the concentration of ABTS radicals can be detected through the absorbance at 734 nm (A_{734 nm}). When an antioxidant (radical scavenger) is added to the ABTS radical solution, the original radicals are neutralized, causing the ABTS radical solution to fade and the absorbance at 734 nm to decrease, thereby evaluating the radical scavenging capacity of the antioxidant.

This test was conducted according to the method described by Arnao et al. (ARNAO MB, CANO A, HERNANDEZ-RUIZ J, GARCIA-CANOVAS F, ACOSTA M (1996) Inhibition by L-ascorbic acid and other antioxidants of the 2,2′-azino-bis(3-ethylbenzothiazoline-6-sulfonic acid) oxidation catalysed by peroxidase: a new approach for determining total antioxidant status of foods. Anal Biochem 236:255-261) with modifications. ABTS (Sigma) was dissolved in deionized water (DI water) at room temperature and the concentration was adjusted to 7 mM. Then, 2.45 mM of potassium persulfate was added, and the mixed solution was placed in a dark room at room temperature to react for 16 hours to generate stable ABTS radical ions (blue-green). The solution was then diluted with deionized water to become an ABTS radical ion working solution with an absorbance of 0.75±0.05 at 734 nm.

Next, the sample to be tested and standard solutions of Trolox at different concentrations were respectively added to the ABTS radical ion working solution, and deionized water alone was used as a control. After reacting for 20 minutes, the absorbance at 734 nm was measured by a spectrophotometer. The scavenging capacity of different concentrations of the standard Trolox solutions for ABTS radical ions was used to plot a scavenging rate standard curve. The absorbance values measured for the test sample and the control group were converted according to this standard curve to obtain the radical scavenging capacity. The radical scavenging capacity represents antioxidant activity, and its calculation formula is shown in the following Formula 1:

$$( 1 - {ABS}_{sample} / {ABS}_{control} ) \times 100 \qquad Formula 1$$

Wherein, ABSₛₐₘₚₗₑ represents the absorbance of the test sample, and ABS_{control} represents the absorbance of the control.

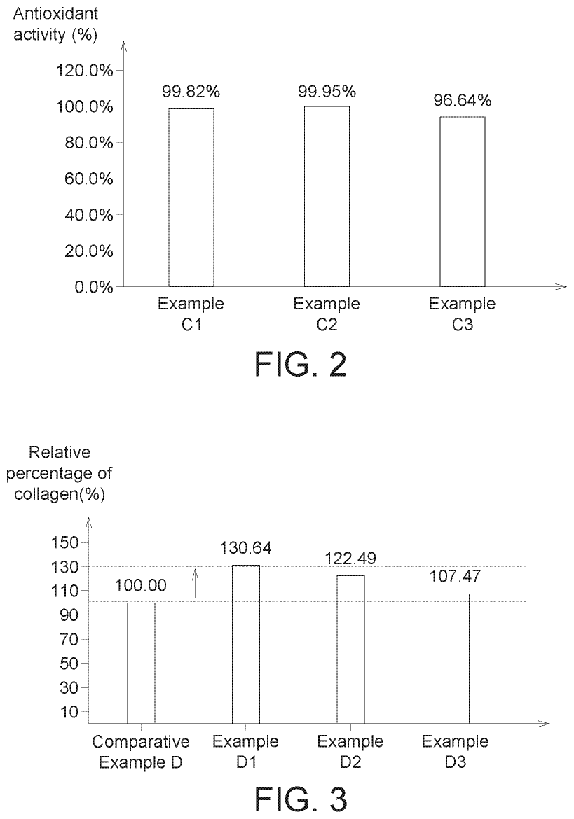

FIG. 2, illustrates the antioxidant activity percentages of the pineapple leaf extracts in Examples C1 to C3 measured by the ABTS radical scavenging assay as described above. The concentrations of the pineapple leaf extracts in Examples C1 to C3 are 2 wt %, 1 wt %, and 0.50 wt %, respectively. From the experimental results, it is shown that the antioxidant activities of Examples C1 to C3 are 99.82%, 99.95%, and 96.64%, respectively. Compared with Example C3, Examples C1 and C2 have higher concentrations of pineapple leaf extract and exhibit better antioxidant activity.

According to some embodiments, the antioxidant capacity of the extract can be evaluated by measuring the total phenolic content (TPC). Generally, a higher total phenolic content of the pineapple leaf extract indicates a stronger antioxidant capacity of the pineapple leaf extract. Refer to Table 1 below, which shows the results of the total polyphenol content and antioxidant activity of Comparative Example A and Examples A1 and A2. Wherein, the total polyphenol content is calculated based on gallic acid equivalent (GAE) and is shown as the total polyphenol content of the pineapple leaf extract per 100 g of pineapple leaves (mg GAE/100 g pineapple leaves). The antioxidant activity was determined by the ABTS radical scavenging assay as described above, and the antioxidant activity in Table 1 represents the ABTS activity at a concentration of 0.5 wt % of the pineapple leaf extract.

Examples A1 and A2 were prepared according to the aforementioned steps S1 to S7 (as shown in FIG. 1). The difference between Comparative Example A and Examples A1 and A2 lies in that Comparative Example A did not undergo the size reduction step (i.e., step S3), while the other processing steps were the same. The average particle size of the pineapple leaf powder obtained in Example A1 after the size reduction step was 0.25 mm, and the average particle size of the pineapple leaf powder obtained in Example A2 after the size reduction step was 0.04 mm.

TABLE 1

| | | Antioxidant activity |
|---|---|---|
| | Total phenolic content (mg | (%)at a concentration |
| | GAE/100 g pineapple | of 0.5 wt % of the |
| Group | leaves) | pineapple leaf extract |
| Comparative | 133.9 | 55.9 |
| Example A | | |
| Example A1 | 180.2 | 67.2 |
| Example A2 | 428.9 | 96.64 |

According to some embodiments, the antioxidant capacity of the extract can be evaluated by measuring the content of specific polyphenols. For example, a higher content of ferulic acid, chlorogenic acid (CGA), and p-coumaric acid (P-CA) in the pineapple leaf extract indicates a stronger antioxidant capacity of the pineapple leaf extract. Refer to Table 2 and Table 3 below, wherein Table 2 shows the contents of ferulic acid and p-coumaric acid of Comparative Example B and Examples B1 to B2, and Table 3 shows the contents of p-coumaric acid and chlorogenic acid of Comparative Example B and Example B3. Examples B1 to B3 were prepared according to the aforementioned steps S1 to S7 (as shown in FIG. 1), wherein Example B1 was sieved through a 0.5 mm mesh, and Examples B2 to B3 were sieved through a 0.08 mm mesh. The difference between Comparative Example B and Examples B1 to B3 lies in that Comparative Example B did not undergo the size reduction step (i.e., step S3), while the other processing steps were the same. Since the average particle size of the pineapple leaf powder obtained in Comparative Example B is larger than the average particle sizes of the pineapple leaf powders obtained in Examples B1 to B3, and the average particle size of the pineapple leaf powder obtained in Example B1 is larger than the average particle sizes of the pineapple leaf powders obtained in Examples B2 and B3, the resulting extracts exhibit corresponding differences in active ingredient content.

TABLE 2

| | | p-Coumaric | total |
|---|---|---|---|
| | Ferulic acid | acid | polysaccharides |
| | (mg/100 g | (mg/100 g | (g/100 g |
| | pineapple | pineapple | pineapple |
| Group | leaves) | leaves) | leaves |
| Comparative | 19.79 | 14.66 | 1.56 |
| Example B | | | |
| Example B1 | 20.24 | 19.74 | 1.72 |
| Example B2 | 22.26 | 21.58 | 2.58 |

TABLE 3

| | Chlorogenic acid |
|---|---|
| | (mg/100 g pineapple |
| Group | leaves) |
| Comparative | 13.88 |
| Example B | |
| Example B3 | 14.62 |

From the results of Tables 1, 2, and 3, it is shown that after the size reduction step, the pineapple leaf extract can have higher contents of total polyphenols, total polysaccharides, ferulic acid, chlorogenic acid, and p-coumaric acid, and the antioxidant activity is also improved. Among them, ferulic acid increased by 12.5%, p-coumaric acid increased by 47.2%, total polysaccharides increased by 65%, and chlorogenic acid also increased by 5.3%, indicating that the extract of the present disclosure possesses enhanced antioxidant capacity. Furthermore, as shown in Table 2, Example B2 has more active ingredients (i.e., total polysaccharides, ferulic acid, and p-coumaric acid) than Example B1, indicating that as the average particle size of the pineapple leaf powder becomes smaller, the extraction of active ingredients may be promoted, thereby increasing the content of active ingredients in the pineapple leaf extract.

### Collagen Production Assay

According to some embodiments, collagen production can be induced by the pineapple leaf extract in a cell-based experiment to evaluate collagen production. The cells used in the experiment were HSF human skin fibroblast cell lines purchased from the Animal Technology Research Institute and were used for this in vitro assay.

The aforementioned HSF human skin fibroblast cell line was seeded in a 6-well cell culture plate at 3×10⁵ cells/well. After the cells were attached, different concentrations of the pineapple leaf extract or other samples were added to the cells for co-culture, and a group without treatment was used as a control group.

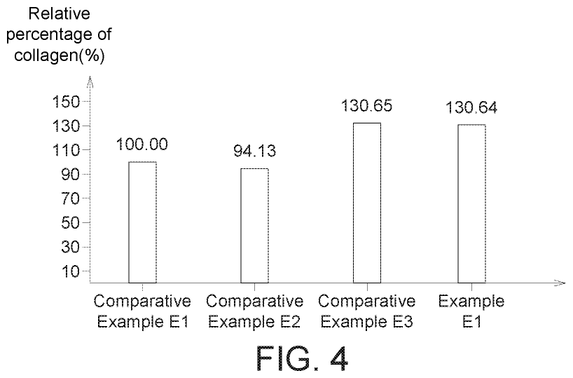

After 7 days of co-culture, the supernatant from each well was collected, and collagen production was quantified using a collagen assay kit (Collagen Assay Kit, specification S1000, brand Biocolor) according to the instructions for use. The analysis results are shown in FIGS. 3 to 4.

FIG. 3 shows the results of collagen production for Comparative Example D and Examples D1 to D3. The Y-axis represents the relative percentage of collagen (the collagen concentration of Comparative Example D is set as 100%, and the collagen concentrations of Examples D1 to D3 are calculated and expressed relative thereto). Comparative Example D (the control group) had no pineapple leaf extract added, and Examples D1 to D3 had 0.2 wt %, 0.5 wt %, and 1.0 wt % of the pineapple leaf extract added, respectively.

As shown in FIG. 3, compared with Comparative Example D, Examples D1 to D3 exhibit increased collagen production.

FIG. 4 shows the results of collagen production for Comparative Examples E1 to E3 and Example E1 measured by the collagen production assay as described above. The Y-axis represents the relative percentage of collagen (the collagen concentration of Comparative Example E1 is set as 100%, and the collagen concentrations of Comparative Examples E2 and E3 and Example E1 are calculated and expressed relative thereto). In Example E1, collagen production was evaluated after treatment with 0.2 wt % of the pineapple leaf extract of the present disclosure. The difference between Comparative Examples E1 to E3 and Example E1 lies in that Comparative Example E1 served as the control group without treatment, Comparative Example E2 was treated with 0.3 wt % histidine, and Comparative Example E3 was treated with 0.2 wt % modified hyaluronic acid (commercially available as HYA-HEAL+).

As shown in FIG. 4, Example E1 has a higher collagen concentration than Comparative Examples E1 and E2, and its collagen concentration is similar to that of Comparative Example E3.

Based on the combined results of FIGS. 3 to 4, it can be seen that the pineapple leaf extract of the present disclosure exhibits increased collagen production in vitro, and its effect is comparable to that of commercially available products (such as modified hyaluronic acid), indicating that the present disclosure has potential value for cosmetic applications and may contribute to improving skin appearance associated with aging.

Table 4 below shows collagen production levels of Comparative Example F and Example F1 measured by the collagen production assay as described above. The pineapple leaf extract of Example F1 was prepared by steps S1 to S7 as described above (as shown in FIG. 1). The difference between Comparative Example F and Example F1 lies in that Comparative Example F did not undergo the size reduction step (i.e., step S3), while the other processing steps were the same. The average particle size of the pineapple leaf powder obtained after the size reduction step in Example F1 was 0.25 mm.

TABLE 4

| | collagen production (relative |
|---|---|
| Group | percentage) |
| Comparative Example F | 74.69 |
| Example F1 | 130.64 |

From the results of Table 4, it can be seen that after the size reduction step, the pineapple leaf extract results in a higher collagen concentration and exhibits increased collagen production.

In summary, the present disclosure provides a cosmetic composition and a method for manufacturing the same. The cosmetic composition comprises a pineapple leaf extract. Experimental results show that the pineapple leaf extract exhibits cosmetic-related benefits, including enhanced antioxidant capacity and increased collagen production.

It will be apparent to those skilled in the art that various modifications and variations can be made to the disclosed embodiments. It is intended that the specification and examples be considered as exemplars only, with a true scope of the disclosure being indicated by the following claims and their equivalents.

1. A method for manufacturing a cosmetic composition, comprising:
preparing a pineapple leaf extract, wherein steps for preparing the pineapple leaf extract comprise:
providing pineapple leaves;
performing a size reduction step on the pineapple leaves to obtain a pineapple leaf powder;
performing an extraction step on the pineapple leaf powder using an aqueous alcohol solution, and collecting an extract resulting therefrom; and
performing a sterilization step on the extract to obtain the pineapple leaf extract.

2. The method for manufacturing the cosmetic composition according to claim 1,
wherein the pineapple leaf powder has an average particle size ranging from 0.01 mm and 0.8 mm.

3. The method for manufacturing the cosmetic composition according to claim 1, wherein the size reduction step is performed using a high-speed centrifugal mill.

4. The method for manufacturing the cosmetic composition according to claim 1, wherein preparing the pineapple leaf extract further comprises a drying step for drying the pineapple leaves.

5. The method for manufacturing the cosmetic composition according to claim 4, wherein the drying step is performed at a temperature of 50° C. to 70° C.

6. The method for manufacturing the cosmetic composition according to claim 4, wherein the pineapple leaves have a moisture content of no greater than 10% after the drying step on a dry weight basis.

7. The method for manufacturing the cosmetic composition according to claim 1, wherein the aqueous alcohol solution is a 30 vol % aqueous ethanol solution.

8. A cosmetic composition manufactured by the method according to claim 1, wherein the cosmetic composition comprises:
1 wt % to 5 wt % of the pineapple leaf extract; and
95 wt % to 99 wt % of water, wherein the pineapple leaf extract has a total polyphenol content between 150 mg GAE/100 g pineapple leaves and 550 mg GAE/100 g pineapple leaves.

9. The cosmetic composition according to claim 8, wherein the pineapple leaf extract comprises at least one active ingredients selected from total polysaccharides, ferulic acid, chlorogenic acid (CGA), p-coumaric acid (P-CA).

10. The cosmetic composition according to claim 9, wherein based on a weight of 100 g of the pineapple leaves, a weight of the total polysaccharides extractable therefrom range from 1 g to 5 g, a weight of the ferulic acid extractable therefrom ranges from 0.1 mg to 30 mg, a weight of the p-coumaric acid extractable therefrom ranges from 0.1 mg to 30 mg, and a weight of the chlorogenic acid extractable therefrom ranges from 0.1 mg to 30 mg.
